(19) United States
(12) Patent Application Publication
(10) Pub. No.: US 2022/0323785 A1
(43) Pub. Date: Oct. 13, 2022

(54) SYSTEMS AND METHODS FOR GAZE-BASED LIGHTING OF DISPLAYS

(71) Applicant: KORRUS, INC., LOS ANGELES, CA (US)

(72) Inventors: RAGHURAM L.V. PETLURI, LOS ANGELES, CA (US); PAUL KENNETH PICKARD, LOS ANGELES, CA (US)

(21) Appl. No.: 17/844,421

(22) Filed: Jun. 20, 2022

Related U.S. Application Data
(63) Continuation of application No. PCT/US20/65964, filed on Dec. 18, 2020.
(60) Provisional application No. 62/950,002, filed on Dec. 18, 2019.

(51) Int. Cl. A61N 5/06; G09G 3/34; G06F 3/01

(52) U.S. Cl. CPC A61N 5/0618; G09G 3/3426; G06F 3/013; G09G 2354/00; G09G 2320/0626; A61N 2005/0659; A61N 2005/0652; A61N 2005/0628

(57) ABSTRACT

A method includes providing a pixelated surface, providing at least one sensor configured to track movement of at least one eye of a user relative to the surface, detecting one or more portions of the surface based on the tracked movement and adjusting one or more properties in relation to the one or more detected portions such that a dosing of one of cyan and long-red, near-infrared (NIR) illumination is provided, while the user is gazing at the one or more portions.

### CROSS REFERENCE TO RELATED APPLICATIONS

This application claims the benefit of U.S. Provisional Patent Appl. No. 62/950,002, filed Dec. 18, 2019 the entire disclosure of which is incorporated herein by reference.

### TECHNICAL FIELD

The present disclosure relates generally to systems and methods for adjusting biological activity via artificial lighting based on an eye focus region (EFR).

### BACKGROUND

Circadian rhythms are biological processes that are generated and regulated by a brain-based biological clock. These biological processes include body temperature, digestion, release of certain hormones, and a person's wake/sleep cycle. In the absence of external cues, circadian rhythms in humans run about every 24 hours. Based on particular light exposures, a person's circadian rhythm may become desynchronized (e.g., with the local day-night cycle).

The circadian system is more sensitive to short-wavelength (blue) light so prolonged exposure to such light can affect various bio-physiological functions.

Light exposure at night can suppress the secretion of the hormone melatonin and can cause people to stay alert, thus delaying an ability to sleep. Many people spend several hours each day in front of a display, which may harm circadian rhythms (e.g., by stimulating blue-light-sensitive ganglion cell photoreceptors), degrade sleep quality, and impair alertness a following day. Certain light can exacerbate development of cataracts, eyelid cancer, pterygium and soft drusen, and age-related macular degeneration (AMD). Visible blue light may even be harmful to the human retina. And children are more severely affected by such media use as watching TV, playing computer games, or looking at online content. There is thus a need to better control light exposure from user devices and to better maintain synchronization or entrainment to a 24 hour cycle.

### SUMMARY

Systems and methods are disclosed for affecting lighting of displays in and/or out of an EFR determined based on one or more output signals of a set of sensors. Accordingly, one or more aspects of the present disclosure relate to a method for: providing a pixelated surface; providing at least one sensor configured to track movement of one or both eyes of the user relative to the surface; detecting one or more portions of the surface based on the tracked movement; and adjusting one or more properties in relation to the one or more detected portions such that a dosing of one of cyan and long-red, near-infrared (NIR) illumination is provided, while the user is gazing at the one or more portions.

The method is implemented by a system comprising one or more hardware processors configured by machine-readable instructions and/or other components. The system comprises the one or more processors and other components or media, e.g., upon which machine-readable instructions may be executed. Implementations of any of the described techniques and architectures may include a method or process, an apparatus, a device, a machine, a system, or instructions stored on computer-readable storage device(s).

### DRAWINGS

The details of particular implementations are set forth in the accompanying drawings and description below. Like reference numerals may refer to like elements throughout the specification. Other features will be apparent from the following description, including the drawings and claims. The drawings, though, are for the purposes of illustration and description only and are not intended as a definition of the limits of the disclosure.

FIG. 1 illustrates an example of a system in which gaze-based lighting is determined, in accordance with one or more exemplary implementations.

FIG. 2 illustrates gamuts and chromaticities of a color space, in accordance with one or more exemplary implementations.

FIG. 3 illustrates a color space, in accordance with the prior art.

FIG. 4 illustrates an example of a system in which backlighting complements a pixelated surface, in accordance with one or more exemplary implementations.

FIG. 5 illustrates an example of a spectral power distribution (SPD) of a two-channel backlight, in accordance with one or more exemplary implementations.

FIG. 6 illustrates different coverage of standard red green blue (sRGB) two-channel lighting based on a delivered equivalent melanopic lux (EML) ratio, in accordance with one or more exemplary implementations.

FIGS. 7A-7F progressively illustrate extreme and intermediate color gamuts of two channel lighting, in accordance with one or more exemplary implementations.

FIG. 8 illustrates an example of an SPD of a four-channel backlight, in accordance with one or more exemplary implementations.

FIG. 9 illustrates an example of an achieved EML ratio based on a white point, in accordance with one or more exemplary implementations.

FIG. 10 illustrates different coverage of sRGB four-channel lighting based on a delivered EML ratio, in accordance with one or more exemplary implementations.

FIG. 11 illustrates an intermediate color gamut of four-channel lighting, in accordance with one or more exemplary implementations.

FIG. 12 illustrates a process for providing gaze-based bioactive lighting, in accordance with one or more exemplary implementations.

### DETAILED DESCRIPTION

As used throughout this application, the word “may” be used in a permissive sense (i.e., meaning having the potential to), rather than the mandatory sense (i.e., meaning must). The words “include,” “including,” and “includes” and the like mean including, but not limited to. As used herein, the singular form of “a,” “an,” and “the” include plural references unless the context clearly dictates otherwise. As employed herein, the term “number” shall mean one or an integer greater than one (i.e., a plurality).

As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other.

Unless specifically stated otherwise, as apparent from the discussion, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device.

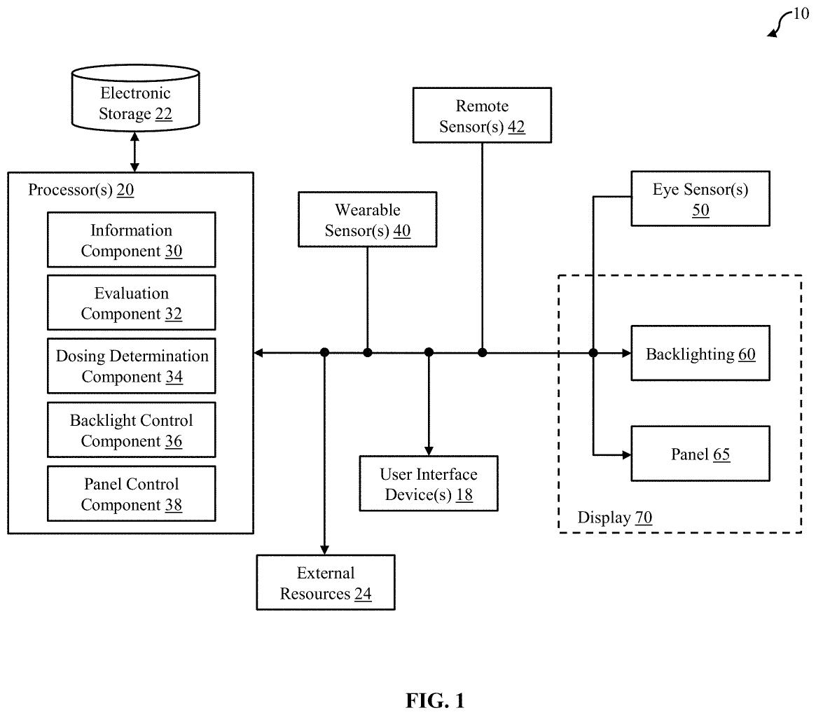

FIG. 1 illustrates a system 10 configured to affect biological activity (e.g., melanoma secretion, pupil diameter, or other measurable attribute) of a user. System 10 may comprise processor 20, electronic storage 22, external resources 24, wearable sensor(s) 40, remote sensor(s) 42, eye sensor(s) 50, and display 70, which may include backlighting 60 and panel 65 (and which may form part of a same device that comprises processor 20). In some exemplary implementations, display 70 may comprise a plurality of displays, including, e.g., left and right screens.

The biological effects of light on humans may be measured in equivalent melanopic lux (EML). Lower EML values from electric lighting may be beneficial in the evening and at night to reduce unwanted health effects linked to melatonin suppression from certain light (e.g., at the wrong time). The term circadian-stimulating energy (CSE) more generally refers herein to any characteristics of a spectral power distribution (SPD) that may biologically affect a subject. For example, backlighting 60 and/or display 70 may generate CSE including one or more of circadian stimulus (CS), circadian illuminance (CLA), EML, blue light hazard (BLH), circadian efficacy of radiation (CER), circadian action factor (CAF), luminous efficacy of radiation (LEF), circadian power, circadian flux, and power of one or more other wavelength ranges. The application of CSE to biological systems (e.g., a mammal or another user) in doses, amount, aliquots, and volumes may be referred to as CSE dosing. CSE dosing may be applied, e.g., with light having a wavelength between 464 and 510 nanometers (nm).

In some instances, exposure to a quantity of blue light may be involved in damage in human eyes. BLH is a known risk and the measure of BLH provides a measure of potential for a photochemical induced retinal injury that results from radiation exposure. Such exposure is one factor which has been linked to photoreceptor damage. It has been reported that the blue light appears to decrease adenosine triphosphate (ATP) energy production in retinal ganglion cells. This has a negative effect on mitochondrial function and oxidative stress which has been shown to decrease survival of ganglion cells. As ganglion cells play a major role in synchronizing circadian rhythms, their destruction inhibits the eye's ability to determine length-of-day and length-of-night. Retinal ganglion cell death further leads to impaired vision. There is also increasing evidence that excessive blue light exposure may cause damage in human skin; it may contribute to wrinkles, worsening skin laxity, and pigmentation issues. When blue light penetrates the skin it can damage DNA, leading to inflammation, the breakdown of healthy collagen and elastin, and hyperpigmentation. It is also reported that excessive blue light at night negatively affects the human body's natural sleep cycle.

Blue light is not the only light in the visible spectrum that can be used to affect biophysiological functions of the human body. Recent studies indicate that therapy which may include doses of long red and near-IR: Long Red typically with a spectrum of >625 nm to <700 nm with peak wavelengths >640-670 nm and NIR typical ranges from >700 nm and <1400 nm (with typical peak wavelengths: 850 nm, 940 nm, 1064 nm) may affect bio-physiological functions by improving eye health, skin health, hair growth, and cognitive function. The spectral sensitivity corresponding to the human eye can be considered to be based on the color-matching functions of the 1931 Standard Observer (XYZ-tristimulus values for CIE 1931 2° color-matching), which show that the effect of light above 700 nm on color perception to be substantially negligible. In other words, it will have no significant impact on the overall (ccx, ccy) color point on the 1931 CIE Chromaticity Diagram of emitted light from a lighting system. In some aspects, the present disclosure relates to long red and near infrared lighting channels that can provide long red and near infrared energy (“LRNE”). Long red and near infrared channels can provide one or both of Visible LRNE and non-visible LRNE. Visible LRNE refers to light having spectral power in wavelengths between about 625 nm and about 700 nm. Non-Visible LRNE refers to light having spectral power in wavelengths greater than or equal to about 700 nm. The long red and NIR channels of the present disclosure can be part of one or more red channels involved in color-tuning and providing white light, or as separate channel that can be operated independently of color-tuning requirements. How the human eye perceives red, long red and near infrared in a given individual may vary based on a plethora of factors including but not limited to age, stimulation of eye before exposure, eye health and health in general. Accordingly, there will be an overlap between the end of long red and the beginning of near infrared. Those of ordinary skill in the art and the skilled artisan will recognize variation is narrow and does not create substantial uncertainty in the terms. Hence the terminology LRNE is encompasses the entirety of both long red and near-infrared.

Additionally, LRNE may be beneficial by reducing, limiting, counteracting or ameliorating some of the negative effects associated with excessive blue light exposure. Disclosed herein are methods and systems to provide therapeutic doses of LRNE either to address a biological condition or as a prophylactic or health supplement means to limit or prevent at least one of an emotional, neurological, immune, and biological condition or system. Bioactive exposure refers to one or both of LRNE and CSE and directing at least one of LRNE and CSE at a biological system which may be a specific organ or any part of the body

The bioactive exposure may be controlled by a control system (described herein) whereby at least one controller, e.g., a computing device receives inputs including fixed, variable and dynamically changing from a variety of sources and the processor associated with the system and method applies at least one of LRNE and CSE in accordance with said control system. Control input data is at least one of input by: users, server, database, derived from a decisioning engine and collected by at least one sensor. The inputs are provided to a processor via signal communication. The processor may be local to the therapeutic device, remote from the therapeutic device or the processing may take place both locally and remote from the therapeutic device. Control systems disclosed herein may adjust the amount and timing of aliquots of bioactive exposure. The control of aliquots and frequency in response to input may be used to dynamically adjust the therapeutic or health supplement application of CSE or LRNE to users. Dynamic adjustment of bioactive exposure to a user may be viewed as personalized whereby data harvested from sensors in the lighting installation environment as well as sensors that reflect information about users, such as one or more of physiological sensors (e.g., sensors 40 and 42). The control system may have modules within the platform which may connect to or integrate with data sources of information about users as described below.

Disclosed herein are additional methods and systems to provide bioactive exposure as one of a supplement and therapeutic dose offline to:

A. Lessen the effect of age-related macular degeneration by stimulating mitochondria in retinal ganglion eye cells to produce more ATP energy. The increase in ATP production has been shown to slow the decline in vision associated with aging. LRNE may additionally improve the effects of glaucoma, a condition that destroys ganglion eye cells, by protecting the cornea and the retina.

B. Address a biological condition or as a prophylactic or supplement means to limit or prevent a biological condition. Examples include but are not limited to, to prevent fluid buildup in the front of the eye, a main complication of glaucoma known to result in cell death of ganglion cells. LRNE has been shown to prevent the death of retinal ganglion cells when the optic nerve has been damaged, thereby preventing vision loss that would otherwise occur.

C. improve skin health and appearance by the application of LRNE therapy. LRNE can reduce acute and chronic inflammation by increasing blood flow to damaged tissues. LRNE may be applied to increase natural collagen production, resulting in younger, healthier looking skin. Rats that were exposed to doses of LRN experienced an increase in collagen synthesis and neoformed bone. Patients dealing with acne or depigmentation conditions, such as vitiligo, may benefit from undergoing LRN therapy, as it can control sebum production (which leads to acne), and it can stimulate melanocyte proliferation (which enhances skin re-pigmentation). Skin that has been wounded, burned, or scarred also repairs more rapidly if it is exposed to LRN, as red light significantly increases tensile strength and wound contraction while decreasing inflammation.

D. A myriad of other bio-physiological function are impacted by LRNEs, including but not limited to, hair growth and cognitive function. LRNE therapy may be used in conjunction with or as an alternative treatment to hormone regulating drugs typically used to treat hair loss. LRNE exposure has been shown to be a treatment in terms of hair regrowth. Research has also demonstrated that LRNE exposure may lead to improved cognitive function with few side effects. In one study, those exposed to LRNE experienced quicker reaction times, better memory, a more positive mood, and the ability to learn new information faster. These beneficial effects on the human brain may be related to LRNE's increasing cerebral blood flow and oxygen availability and boost ATP energy production.

E. LRNE therapy may be able to counteract, limit or ameliorate the negative effects from excessive CSE and blue light exposure. When humans absorb natural blue light from the sun, they also absorb natural red light from the sun, the two together providing numerous health benefits. However, an overload of artificial blue light such as CSE by itself may be determinantal. This damage can be mitigated through LRN exposure.

In some exemplary implementations, backlighting 60 may form part of a set of integrated circuits (ICs). In some exemplary implementations, backlighting 60 may be implemented, e.g., using light-emitting diodes (LEDs), organic LEDs (OLEDs), cold cathode fluorescent lamps (CCFL), mini LEDs, micro LEDs, or another suitable light source. In some exemplary implementations, backlighting 60 may implement direct backlighting (also known as full-array), e.g., with LEDs placed behind panel 65. In other implementations, backlighting 60 may be edge-lit, e.g., with LEDs positioned along opposing sides of a screen.

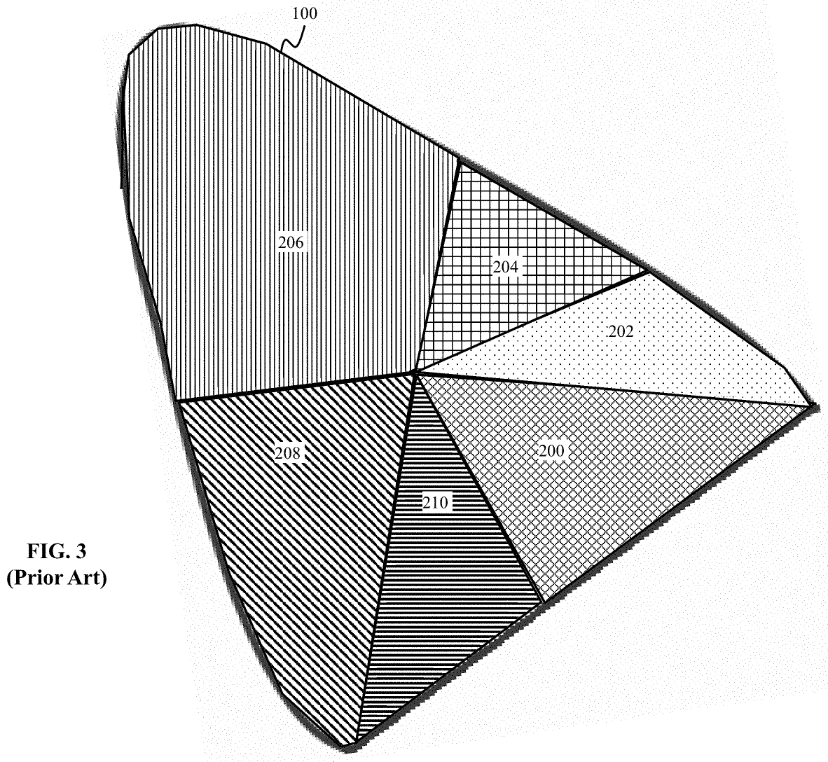

Chromaticity is an objective specification of the quality of a color regardless of its luminance. Chromaticity may be characterized by hue and colorfulness (or saturation) parameters. Light emitted by display 70 may be represented by points plotted on a chromaticity diagram, such as the 1931 international commission on illumination (CIE) chromaticity coordinate system exemplarily depicted in FIG. 3. Useable color spaces may include the 1976 CIELUV, the CIE 1931 red green blue (RGB) color space, and/or the CIE 1931 XYZ color spaces. A region on a chromaticity diagram may represent light sources having similar chromaticity coordinates. For example, FIG. 3 depicts generally red region 200, generally orange region 202, generally yellow region 204, generally green region 206, generally blue region 208, and generally purple region 210; but these are merely simplistic generalizations, as visible light comprising these colors is known to be continuously spread across the corresponding wavelengths. That is, chromaticity coordinates scale 100 may comprise a range of wavelengths, e.g., between 360 and 780 nm.

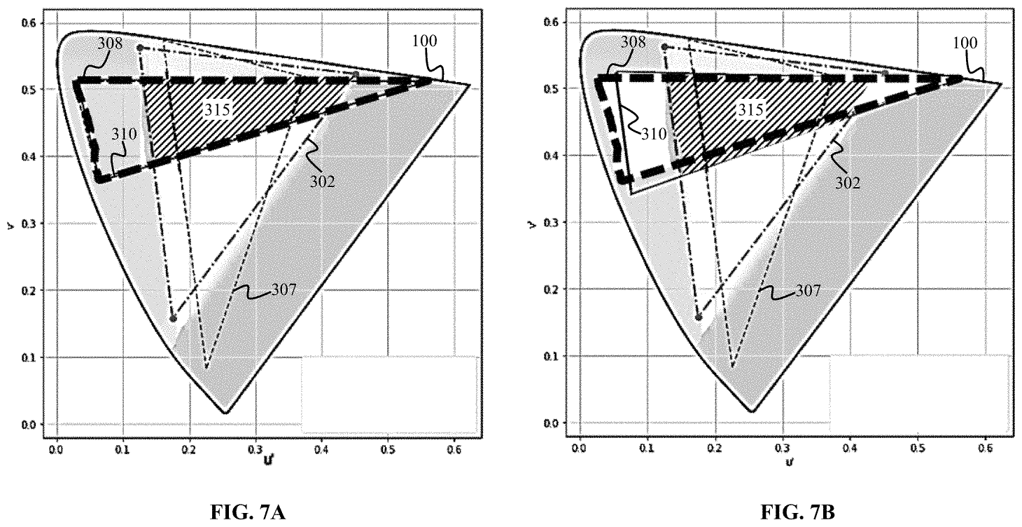

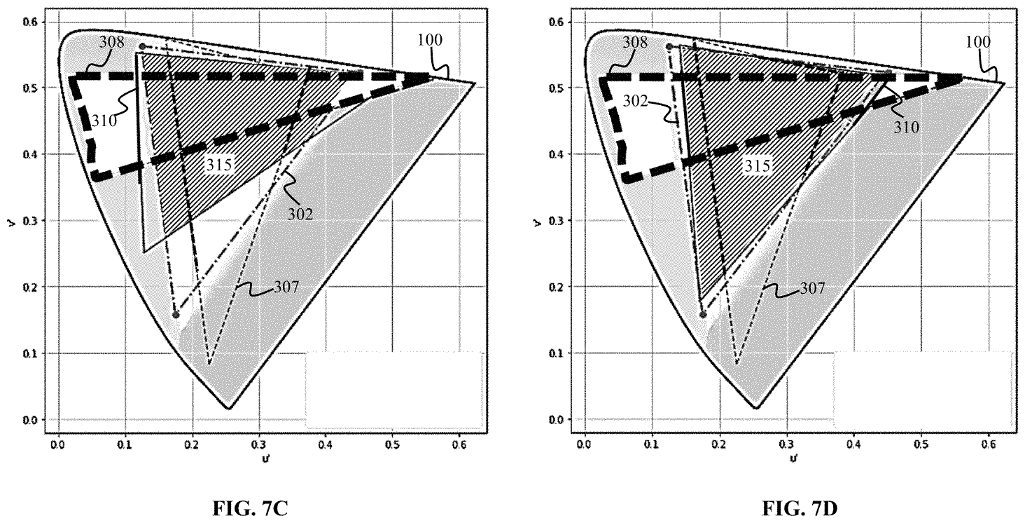

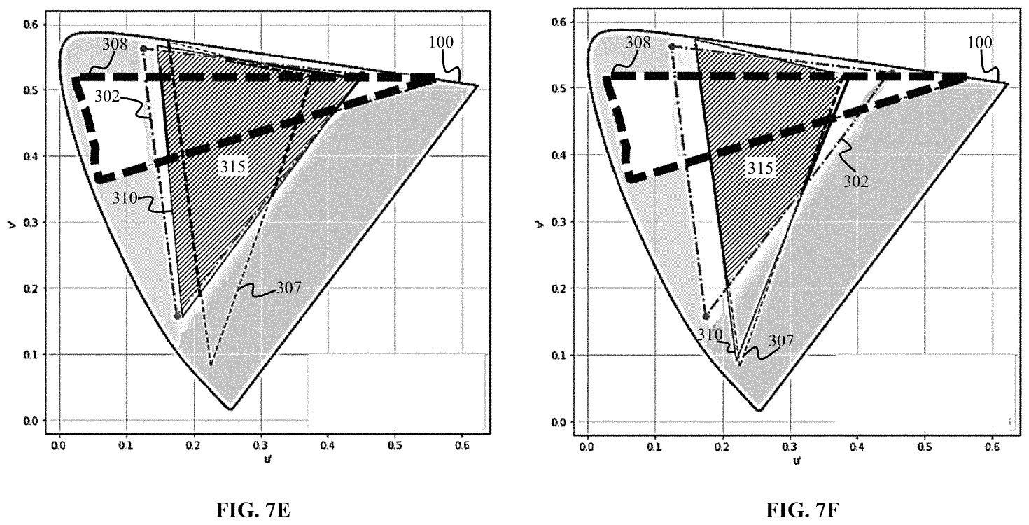

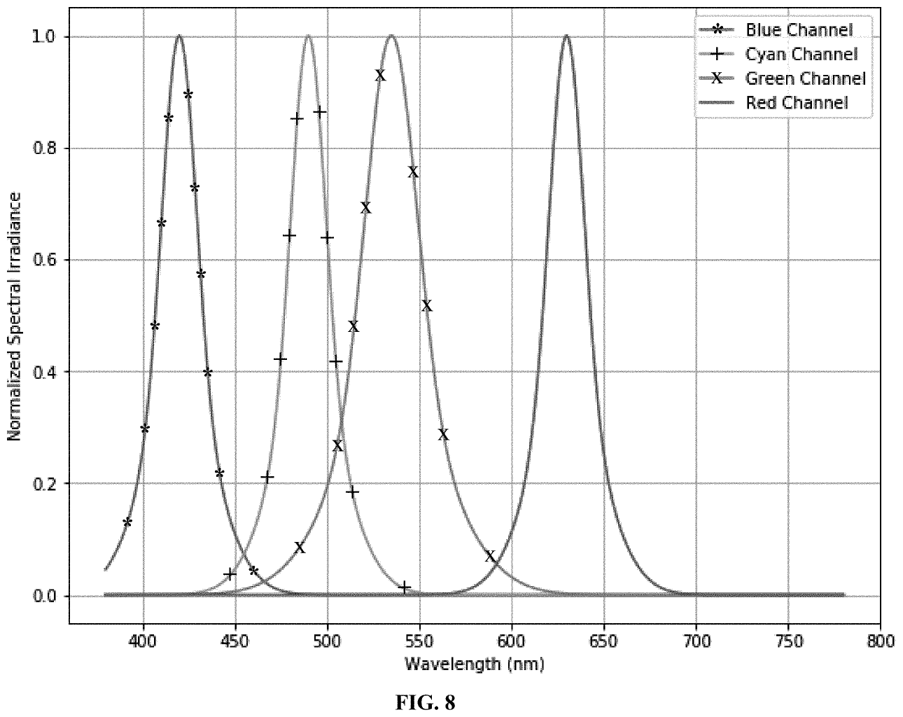

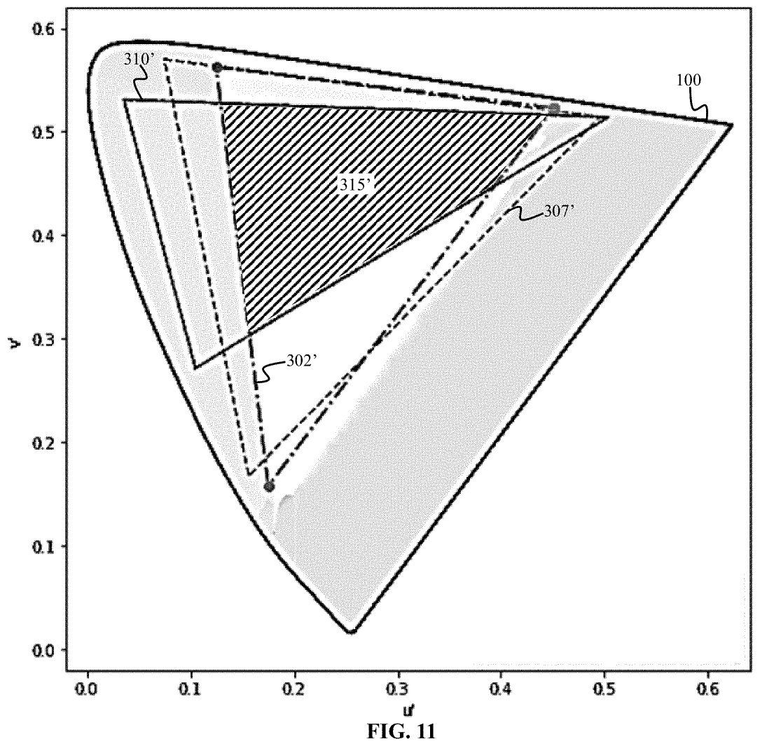

The 1931 CIE chromaticity diagram maps out the human color perception in terms of parameters x and y, these parameters also being referred to as u′ and v′, respectively, as depicted in FIGS. 7 and 11. The spectral colors are distributed around the edge of outline 100, which includes all of the hues perceived by the human eye. Outline 100 represents maximum saturation for the spectral colors, and the interior portion represents less saturated colors, including white light.

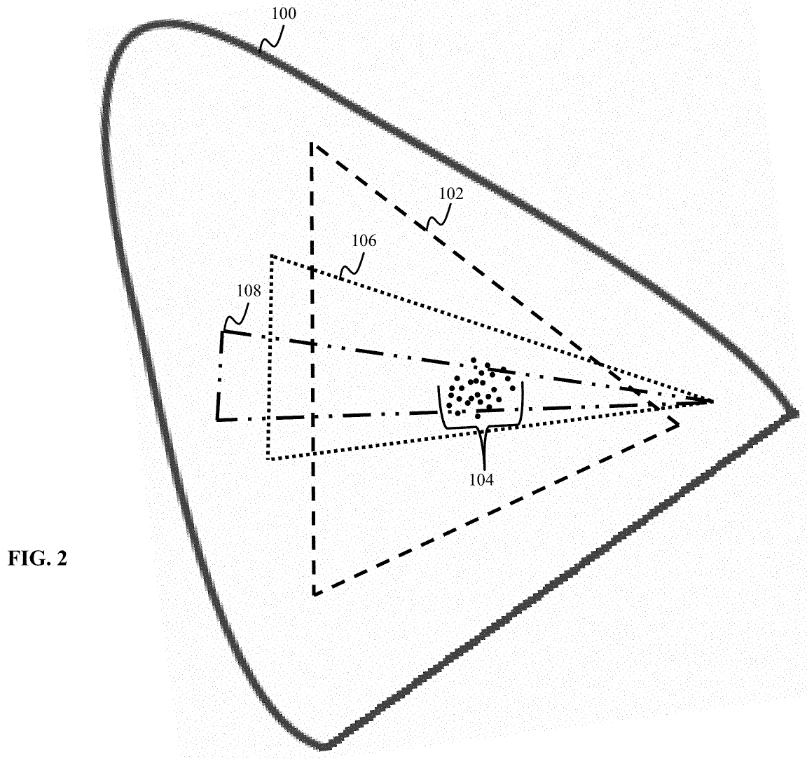

Chromaticity coordinates scale 100 may enclose sRGB triangle 102, triangle 106, triangle 108, and cluster 104, as depicted in FIG. 2. Cluster 104 may include a plurality of color chromaticities each representing a different pixel in a segment of a screen that corresponds to a single LED (e.g., one LED 62) of backlight 60. Triangle 102 may comprise a typical portion of a color space that is covered by a standard sRGB monitor. Triangle 106 may comprise a minimum gamut that encloses cluster 104. And triangle 108 may comprise a high EML gamut. A color gamut is the entire range of colors and tones achievable, e.g., from RGB to CMYK colors.

Although not shown in FIG. 2, a lower or lowest EML gamut would be another triangle either the same as sRGB triangle 102 or a larger triangle that encloses sRGB triangle 102. In some exemplary implementations, display 70 may be configured to generate a blend between a highest EML gamut and a lowest EML gamut, e.g., with 50% being a low EML gamut and 50% being a high EML gamut, which would effectively result in a triangle that fits somewhere in the middle. Triangle 106 exemplarily depicts one such blend.

In some exemplary implementations, one or more components of processor 20 may be embedded in a processing device (e.g., a graphics processing unit (GPU)) of display 70. For example, RGB pixel values may be received from processor 20, from a GPU, or from another processor embedded in display 70.

Electronic storage 22 of FIG. 1 comprises electronic storage media that electronically stores information. The electronic storage media of electronic storage 22 may comprise system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., user interface (UI) device 18, processor 20, etc.). In some exemplary implementations, electronic storage 22 may be located in a server together with processor 20, in a server that is part of external resources 24, in UI devices 18, and/or in other locations. Electronic storage 22 may comprise a memory controller and one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information obtained and/or determined by processor 20, information received via UI devices 18 and/or other external computing systems, information received from external resources 24, and/or other information that enables system 10 to function as described herein.

External resources 24 may include sources of information (e.g., databases, websites, etc.), external entities participating with system 10, one or more servers outside of system 10, a network, electronic storage, equipment related to Wi-Fi technology, equipment related to Bluetooth® technology, data entry devices, a power supply (e.g., battery powered or line-power connected, such as directly to 110 volts AC or indirectly via AC/DC conversion), a transmit/receive element (e.g., an antenna configured to transmit and/or receive wireless signals), a network interface controller (NIC), a display controller, a GPU, and/or other resources. In some implementations, some or all of the functionality attributed herein to external resources 24 may be provided by other components or resources included in system 10. Processor 20, external resources 24, UI device 18, electronic storage 22, a network, and/or other components of system 10 may be configured to communicate with each other via wired and/or wireless connections, such as a network (e.g., a local area network (LAN), the Internet, a wide area network (WAN), a radio access network (RAN), a public switched telephone network (PSTN), etc.), cellular technology (e.g., GSM, UMTS, LTE, 5G, etc.), Wi-Fi technology, another wireless communications link (e.g., radio frequency (RF), microwave, infrared (IR), ultraviolet (UV), visible light, cm wave, mm wave, etc.), a base station, and/or other resources.

UI device(s) 18 of system 10 may be configured to provide an interface between one or more users and system 10. UI devices 18 are configured to provide information to and/or receive information from the one or more users. UI devices 18 include a user interface and/or other components. The UI may be and/or include a graphical UI (GUI) configured to present views and/or fields configured to receive entry and/or selection with respect to particular functionality of system 10, and/or provide and/or receive other information. In some exemplary implementations, the UI of UI devices 18 may include a plurality of separate interfaces associated with processors 20 and/or other components of system 10. Examples of interface devices suitable for inclusion in UI device 18 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that UI devices 18 include a removable storage interface. In this example, information may be loaded into UI devices 18 from removable storage (e.g., a smart card, a flash drive, a removable disk) that enables users to customize the implementation of UI devices 18.

In some exemplary implementations, UI devices 18 are configured to provide a UI, processing capabilities, databases, and/or electronic storage to system 10. As such, UI devices 18 may include processors 20, electronic storage 22, external resources 24, and/or other components of system 10. In some exemplary implementations, UI devices 18 are connected to a network (e.g., the Internet). In some exemplary implementations, UI devices 18 do not include processor 20, electronic storage 22, external resources 24, and/or other components of system 10, but instead communicate with these components via dedicated lines, a bus, a switch, network, or other communication means. The communication may be wireless or wired. In some exemplary implementations, UI devices 18 are laptops, desktop computers, smartphones, tablet computers, and/or other UI devices.

Data and content may be exchanged between the various components of the system 10 through a communication interface and communication paths using any one of a number of communications protocols. In one example, data may be exchanged employing a protocol used for communicating data across a packet-switched internetwork using, for example, the Internet Protocol Suite, also referred to as TCP/IP. The data and content may be delivered using datagrams (or packets) from the source host to the destination host solely based on their addresses. For this purpose the Internet Protocol (IP) defines addressing methods and structures for datagram encapsulation. Of course other protocols also may be used. Examples of an Internet protocol include Internet Protocol Version 4 (IPv4) and Internet Protocol Version 6 (IPv6).

In some exemplary implementations, processor(s) 20 may be communicable coupled to display 70. In some exemplary implementations, processor(s) 20 and/or display 70 may each form part (e.g., in a same or separate housing) of a user device, a consumer electronics device, a mobile phone, a smartphone, a personal data assistant, a digital tablet/pad computer, a wearable device (e.g., watch), augmented reality (AR) goggles, virtual reality (VR) goggles, a reflective display, a personal computer, a laptop computer, a notebook computer, a work station, a server, a high performance computer (HPC), a vehicle (e.g., embedded computer, such as in a dashboard or in front of a seated occupant of a car or plane), a game or entertainment system, a set-top-box, any luminaire, a monitor, a television (TV), a panel, a space craft, or any other device. A housing, which may comprise within its processor(s) 20 and/or display 70, may include or exclude eye sensor(s) 50 configured to determine a gaze or EFR for a user by tracking movement of one or both of his or her eyes relative to a pixelated surface of panel 65.

In some exemplary implementations, processor(s) 20 may be configured to provide information processing capabilities in system 10. Processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some exemplary implementations, processor 20 may comprise a plurality of processing units. These processing units may be physically located within the same device (e.g., a server), or processor 20 may represent processing functionality of a plurality of devices operating in coordination (e.g., one or more servers, UI devices 18, devices that are part of external resources 24, electronic storage 22, and/or other devices).

As shown in FIG. 1, processor 20 is configured via machine-readable instructions to execute one or more computer program components. The computer program components may comprise one or more of information component 30, evaluation component 32, dosing determination component 34, backlight control component 36, panel control component 38, and/or other components. Processor 20 may be configured to execute components 30, 32, 34, 36, and/or 38 by: software; hardware; firmware; some combination of software, hardware, and/or firmware; and/or other mechanisms for configuring processing capabilities on processor 20.

It should be appreciated that although components 30, 32, 34, 36, and 38 are illustrated in FIG. 1 as being co-located within a single processing unit, in exemplary implementations in which processor 20 comprises multiple processing units, one or more of components 30, 32, 34, 36, and/or 38 may be located remotely from the other components. For example, in some exemplary implementations, each of processor components 30, 32, 34, 36, and 38 may comprise a separate and distinct set of processors. The description of the functionality provided by the different components 30, 32, 34, 36, and/or 38 described below is for illustrative purposes, and is not intended to be limiting, as any of components 30, 32, 34, 36, and/or 38 may provide more or less functionality than is described. For example, one or more of components 30, 32, 34, 36, and/or 38 may be eliminated, and some or all of its functionality may be provided by other components 30, 32, 34, 36, and/or 38. As another example, processor 20 may be configured to execute one or more additional components that may perform some or all of the functionality attributed below to one of components 30, 32, 34, 36, and/or 38.

In some exemplary implementations, one or more aspects of display 70 may be based on Eidophor, electroluminescence, electronic paper, LED, LCD (e.g., twisted nematic (TN), inplane switching (IPS), advanced fringe field switching (AFFS), multi-domain vertical alignment (MV A), patterned vertical alignment (PV A), advanced super view (ASV), plane line switching (PLS), and TFT dual-transistor pixel (DTP) or cell technology), cathode-ray tube (CRT), plasma, digital light processing (DLP), liquid crystal on silicon (LCoS), OLED, microLED, organic light emitting transistor (OLET), surface-conduction electron-emitter, field emission, laser TV, microelectromechanical system (MEMS), quantum dot, ferro liquid crystal, thick-film dielectric electroluminescence, telescopic pixel, and/or laser-powered phosphor technology. In some exemplary implementations, evaluation component 32 may use one or more output signals of sensor(s) 50 to determine that a user is gazing at a pixelated surface of one or more of these display devices to read text. Accordingly, whereas some exemplary implementations may be directed to merely providing health-promoting light, other implementations may further provide reading-comprehension-promoting light.

In some exemplary implementations, eye sensor 50 may comprise one or more cameras, a projector, and corresponding control software. As such, sensor 50 may project a region of panel 65 whereat the user's gaze is focused, e.g., to determine which set of pixels the user is viewing. For example, the projector may create a pattern of light (e.g., infra-red or NIR light) on the eyes, and then the camera may take images of the user's eyes with the pattern. The algorithm (e.g., which may include machine-learning or other image processing techniques) may determine the EFR. In another example, eye sensor(s) 50 may comprise a first light source operable to emit light of a first type, a second light source operable to emit light of a second type, the one or more cameras operable to capture an image of a user's eye and reflections of the light of the first type from the user's eye, a primary lens associated with the one or more cameras, an aperture device positioned between the one or more cameras and the primary lens, and evaluation component 32 in communication with the first light source, the second light source, and the one or more cameras. Evaluation component 32 may be configured to control the first light source, the second light source, and the one or more cameras and to process the captured image to detect a gaze direction of the user. The light of the first type may be a ray and directed towards a user's eye, when display 70 is worn by the user. The light of the second type may be a ray and used to diffusely illuminate the user's eye, when display 70 is worn by the user. Light of the first and second type may be pulses and/or of different wavelengths. The primary lens or another optical element may be used to direct reflections of light from the user's eye towards the one or more cameras, the lens or other optical element being positioned on an optical path of the reflected light between the user's eye and the one or more cameras. In some examples, sensor 50 may be used to implement bright pupil eye tracking and dark pupil eye tracking.

A user's EFR with respect to a screen of display 70 may be based on a specified location (e.g., via a pointing device communicable to processor 20 via UI device 18). Otherwise, eye sensor 50 may be used to track the user's eye's position and movement. While gazing at an EFR, backlight control component 36 and/or panel control component 38 may configure display 70 to produce certain (e.g., high) resolution imaging, certain white (e.g., at about 6,500 K) light, and a certain (e.g., high EML) gamut within this EFR. These component(s) may further cause display 70 to generate certain dosing colors around and outside of the EFR (e.g., lower EML gamut). In other implementations, these certain dosing colors may be provided inside the EFR.

Each EFR may correspond with a center of a user's eye's retina, the fovea. The back of a user's eye has a retina, which contains millions of light receptors that convert light into electrified signals that are sent to vision centers of the brain. The retina contains two major categories of photoreceptors called cones and rods because of their geometric shapes. The very central part of the retina, called the fovea, contains only cones.

More particularly, some exemplary implementations of dosing component 34 may determine a dosing of long red NIR energy (LRNE) inside an EFR and of CSE outside the EFR. In other exemplary implementations, dosing component 34 may determine a dosing of LRNE outside an EFR and of CSE inside the EFR. In other exemplary implementations, dosing component 34 may determine a dosing of LRNE outside an EFR and of CSE inside the EFR. In other exemplary implementations, dosing component 34 may determine a dosing of both LRNE and CSE inside an EFR. And in other exemplary implementations, dosing component 34 may determine a dosing of both LRNE and CSE outside an EFR.

In some exemplary implementations, panel 65 may comprise a pixelated surface. For example, this surface may have addressable subsections of a screen that may be controlled to respond to a determined EFR. Panel 65 may implement foveated imaging, which is a digital image processing technique that varies the image resolution or amount of detail across the image according to one or more EFRs. An EFR may correspond to a user's fovea, and its location may be specified in different ways. For example, eye sensor 50 may precisely measure the eye's position and movement to determine the EFR. In another example, eye sensor 50 may cause a projection of the foveal region of the retina of the user's eye. In another example, eye sensor 50 may measure rotation or movement of the eye, e.g., via measurement of the movement of an object (e.g., a form of contact lens) attached to the eye, optical tracking without direct contact to the eye, or measurement of electric potentials using electrodes placed around the eyes.

Pixels of panel 65 may be manufactured into any suitable technology. Each pixel may comprise one, two, or more bands. Each band may have a certain color depth or bit depth. For example, an RGB color-based image has 3 bands, the red band (R), the green band (G) and the blue band (B). Each of the R, G and B bands may have a depth of 8 bits or more. Hence, in this example, each pixel may have a total bit depth of 24 bits or more. In another example, an infrared (IR) image has I-band, the IR-band. This band may have a bit depth of 12-bits. For the purpose of computational convenience, it may be stored within 16-bits. Hence, in this example, each pixel may have a total bit depth of 16-bits.

As mentioned, display 70 may comprise an AR or VR system. Such exemplary implementations may be transmissive or reflective. In some exemplary implementations, AR display 70 may be implemented via waveguides, micro prisms, cascade coated mirrors, or retinal lasers. For example, the AR may comprise diffractive waveguides or reflective waveguides. The AR or VR system may perform optical projection and interface with handheld devices. This system may be a headset, head-mounted display (HIVID), eyeglasses, contact lenses, virtual retinal display, or another suitable fixture.

In some exemplary implementations, wearable sensor 40 may form part of a pendant, an armband, a wrist band, a dongle, a tag, a watch, a chest band, glasses, clothing, a headset, an activity tracker, and the like.

In some exemplary implementations, remote sensor 42 may include one or more ambient sensors of the user's environment (e.g., car, office, room, shower, etc.) to collect information about the actual lighting conditions (e.g., room lighting and/or seasonal lighting conditions) in the environment, activities of occupants within the environment, and the like.

Any of the herein disclosed sensors may be implemented via wearable sensor(s) 40 and/or via remote sensor(s) 42. For example, these sensors may include one or more of a light exposure sensor, motion sensor, temperature sensor, video camera, IR sensor, microwave sensor, LIDAR, microphone, olfactory sensor, haptic sensor, bodily secretion sensor (e.g., pheromones), ultrasound sensor, and/or another sensing device.

In some exemplary implementations, eye sensor 50 may track a gaze of a user with respect to a monitor, VR headset, AR headset, or another (e.g., wearable) device. In other implementations, eye sensors 50 may be installed in a shower such that gaze of a user is tracked while bathing, the tiles of the shower being configured to emit beneficial light, as disclosed herein, based on this gazing. In some exemplary implementations, eye sensor 50 may be integrated into display 70 to detect the current orientation of the user's head and the direction of the user's gaze. For example, the orientation of the user's head may be captured using optical sensors and accelerometers while the current direction of the user's gaze may be captured with optical eye tracking devices such as cameras. In this or another example, eye sensor 50 may provide the user's current view to processor 20, which may then adjust graphics processing accordingly (e.g., to ensure that the current image frames being rendered are based on the current perspective of the user). The line of gaze of an eye corresponds to the optical axis of the eye, while the desired line of sight is determined by the retinal position (e.g., of a slightly off-axis fovea). The line of sight may be estimated from the line of gaze using an estimate of the position of the fovea. The position of the fovea may either be assumed (e.g., based on population data obtained via electronic storage 22) or estimated via calibration. In some exemplary implementations, eye sensor 50 may be explicitly calibrated, e.g., requiring the user to fixate on a set of targets, or it may be implicitly calibrated, e.g., relying on inferring when the user is fixating on known scene points. Calibration may be performed anew each viewing session, or calibration data may be stored and retrieved when the user interacts with display 70.

In some exemplary implementations, system 10 may be designed for an operation that is coordinated with one or more external systems, e.g., room lighting, sound equipment, video and other entertainment systems, weather systems, climate systems, collective mood indicators (e.g., based on stock market data, news feeds, or sentiment indices), analyses of social network data, and other computer systems. In some exemplary implementations, display 70 may be configured to simulate a sunrise, a seasonal affective disorder (SAD) lamp, and/or a downlight. For SAD, the standard recommendation is 10,000 lux of 30 minutes. But some exemplary implementations may result in effective treatment (e.g., of SAD or in supporting circadian rhythms) with as low a light level as 100 lux (e.g., blue light) for 20 minutes.

In some exemplary implementations, display 70 may be controlled by processor 20, which can communicate various lighting levels, timing, and configuration, e.g., to achieve the desired bioactive lighting. Such display properties may vary based on one or more of a determined time of day, a determined geolocation of display 70 at this time, an intended effect of the lighting, an estimated body clock of the user, individual preferences, capabilities of the underlying device, a feedback mechanism, sensor input, and/or another factor.

Display 70 may be used treat or otherwise affect a user's biological system and cycles of the exposed user throughout the day in different ways. For example, backlight control component 36 and/or panel control component 38 may automatically, semi-automatically, or manually adjust the user's light exposure (e.g., based on sensor data, activity data, social media data, etc.). As such, system 10 may be an autonomous control system that automatically adjusts display parameters. For example, system 10 may include an operational feedback system based on a collection of information about the actual lighting conditions (e.g., soliciting and receiving user feedback and/or desired changes).

Inputs from wearable devices may be used in the operational feedback system, such as to measure reactions to lighting conditions (such as to enable automated adjustment of a lighting installation), as well as to measure impacts on mood, health conditions, energy, wellness factors, and the like.

In some exemplary implementations, information component 30 may obtain input information from one or more of users (e.g., via UI device 18), a server (e.g., accessible via external resources 24), a database (e.g., electronic storage 22), a decisioning engine (e.g., a component of processor 20), and a sensor (e.g., sensors 40, 42, and/or 50). For example, information component 30 may obtain live speech or stored voice recordings such that evaluation component 32 assesses a user's tone or mood and that dosing determination component 34 adjusts a lighting dose based on the same.

In some exemplary implementations, information component 30 may obtain data (e.g., physiological) about a user, via wearable sensor(s) 40 and/or remote sensor(s) 42 and/or via UI device 18. This data may include an acceleration of the user, a location of the user (e.g., GPS based or via another positioning system), an orientation or angular velocity (e.g., gyroscope-based) of the user, ambient light characteristics to which the user is exposed, steps walked by the user, a sleep history of the user, a heart rate of the user, a blood pressure of the user, a room temperature, a personal temperature, oxygen saturation of the user, activity type of the user, activity level of the user, galvanic skin response, respiratory rate, cholesterol level, a barometric pressure, localized lighting conditions, lighting spectrum characteristics, humidity, UV light, sound (e.g., ambient noise measured in decibels), particles, pollutants, gases, radiation, hormonal or adrenal levels of the user (e.g., cortisol, thyroid, adrenaline, melatonin, and others), histamine levels, immune system characteristics, blood alcohol levels, drug content, macro and micro nutrients, mood, emotional state, alertness, sleepiness, and/or other attributes related to the user. As such, some exemplary implementations of dosing determination component 34 may manage doses across a plurality of lighting data, including usage of a desk lamp, work monitor, home monitor, mobile phone, smart glasses, and/or overhead office bulbs. This management may even be based on such factors as ambient sound levels and health metrics (e.g., blood pressure, stress level, etc.) of the user.

In some exemplary implementations, information component may obtain social media data related to users, e.g., including social networks (e.g., Facebook™, Linkedln™, Twitter, and the like), sources of medical records (e.g., 23&Me™ and the like), productivity, collaboration and/or calendaring software (e.g., Google™, Outlook™, scheduling apps and the like), information about web browsing and/or shopping activity, activity on media streaming services (e.g., Netflix™, Spotify™, YouTube™, Pandora™ and the like), health record information and other sources of insight about the preferences or characteristics of users of display 70, including psychographic, demographic, and other characteristics. Accordingly, emissions from display 70 may be based on previous exposure(s) to light by a user, one or more demographics (e.g., ethnicity) of the user, and/or one or more other demographics (e.g., an age, including children of teen years and/or younger, who may suffer from greater melatonin suppression even when exposed to a same set of lighting) of the user.

In some exemplary implementations, dosing component 34 may determine a dosing based on any data obtained by information component 30. This data may be user-supplied (e.g., via UI device 18) parameters, such as personal information (e.g., sex, age, etc.), health goals, and light emission targets.

In some exemplary implementations, evaluation component 32 may determine one or more portions of panel 65 at which a user is gazing (also referred to herein as the EFR) based on eye-movement tracked by eye sensor 50.

In some exemplary implementations, evaluation component 32 may measure, via wearable sensor 40 and/or remote sensor 42, physical activity, ambient noise, a hormonal level, and/or an insulin level, with respect to the user. Dosing determination component 34 may then adjust one or more display properties based on these measurement(s) satisfying one or more criteria, while the user is gazing at an EFR. For example, one such criterion may be a noise threshold above which stress of the user increases and sleep is liable for disruption.

In some exemplary implementations, evaluation component 32 may measure, via wearable sensor 40 and/or remote sensor 42, an exposure of the user to a cyan wavelength over at least one first time frame and/or an exposure of the user to an LRNE wavelength over at least one second time frame. Dosing determination component 34 may then adjust one or more display properties based on the measurement(s) satisfying one or more criteria, while the user is gazing at the EFR. The contemplated LRNE wavelengths may be the same as the ones listed in Tables A-1, A-2, and/or A-3 of International Patent Application No. PCT/US2019/060634, the entire contents of which are incorporated herein by reference.

In some exemplary implementations, evaluation component 32 may train a machine learning model using much sensor data accumulated from many different users or from much data of a same user. This model may learn patterns from the sensors' outputs to better determine operating parameters associated with display 70.

The circadian system is very sensitive to short-wavelength (blue) light, with a peak spectral sensitivity at around 460 nm. In some exemplary implementations, dosing component 34 may determine a dosing comprising long blue light, with a wavelength of 480 nm to 490 nm. As such, dosing determination component 34 may replace the harmful blue light with beneficial blue light (and/or blue-enriched-white light), e.g., during the day when melatonin levels are naturally low. Some benefits to the user of this or other provided light may include better memory consolidation, alertness, vigilance, and retention of verbal material. Dosing determination component 34 may help to cause a person to reset their biological clock.

In some exemplary implementations, dosing determination component 34 may generate a certain band of blue and/or ultraviolet that causes molecules in the user's skin to break down into nitric oxide for reducing blood pressure. For example, dosing component 34 may determine a dosing that comprises CSE and/or LRNE based on an evaluation of component 32. In some exemplary implementations, dosing determination component 34 may generate a dosing that comprises cyan, with a wavelength of 490 nm to 520 nm. In these or other implementations, dosing determination component 34 may generate a (e.g., long) red light that helps with cellular regeneration.

In some exemplary implementations, dosing component 34 may determine dosing based on one or more physiological factors obtained by information component 30. These factors may include health conditions, emotional states, moods, energy, wellness factors, and/or another characteristic.

In some exemplary implementations, dosing component 34 may determine dosing for a user that balances exposure of both artificial blue light and LRNE, e.g., to support wellness benefits similar to those from natural, sunlight exposure. For example, certain blue wavelengths of light may decrease blood pressure, increase blood flow, and improve overall endothelial function. As a result, systolic blood pressure and vascular resistance have been shown to decrease.

In some exemplary implementations, dosing component 34 may determine dosing of light to be provided at eye(s) of a user, e.g., with a maximum of 580 lux (lx). In some exemplary implementations, dosing component 34 may determine dosing comprising different combinations of bands (e.g., which may be more beneficial than each individually), including different combinations of (I) visible light, (ii) IR, (iii) NIR, (iv) long or deep blue, and (v) cyan. One or more of these bands may be generated from a secondary emitter (e.g., IR and/or UV emittance) different from emissions of display 70.

In some exemplary implementations, display 70 may be configured to emit one or more of LRNE and CSE, in a range from constant-on to a set of micro-pulses each with a duration less than 1.0 or 0.1 seconds (s). Light pulses may be used to provide bioactive exposure to a set of users. One or more of such pulses may have a frequency between 10 Hz and 0.5 MHz an amount of emitted pulses may vary from a single pulse up to 400,000 pulses (or more). The SPD and intensity may each remain constant or vary, during this or other set of pulse-train emissions. In some exemplary implementations, dosing component 34 may determine a pulse train comprising any suitable waveform of light (e.g., short duration pulses, long duration pulses, square waves, sine waves, based on a variable signal, and/or based on another pattern).

The portion of dosing determination component 34, which determines emissions of light pulse trains, may be a standalone device that includes an emitter. In other exemplary implementations, a light-pulse emitter may be integrated into display 70.

The herein disclosed light pulse trains may be gaze-based and used in an active phase shifting (e.g., to deal with jet lag, whether before, during, or after a time-zone shift), a personal device (e.g., to adjust an individual), an aircraft lighting system (e.g., to adjust the passenger and crew to a destination time-zone), a mental health treatment (e.g., to treat SAD, depression, ADHD, Alzheimer's, autism, or another disease), supporting normal circadian rhythms in healthy population, stabilization of rhythms using closed loop control (e.g., when integrated with biosensors), hospitals, health and wellness, space stations, space craft (e.g., in manned trips to Mars or any other extra-terrestrial place) that lack in a normal 24 hour light-dark cycle, and easing people out of bed (e.g., by suppressing melatonin before they need to wake up while not disrupting their sleep). In some exemplary implementations, dosing component 34 may determine emissions that provide benefits better than nature (e.g., via pulses and other lighting approaches that perform better than mere sunlight exposure, such as by balancing and/or controlling an exposure of both artificial blue light and LRNE to support wellness benefits).

A body's circadian system is optimally sensitive to short pulses of light, with fairly long periods of darkness in between. The herein disclosed light pulse trains may match or even exceed the phase shifting abilities of continuous light, when matched for intensity. In some configurations, the duty cycle may be as low as 1/100,000, effectively minimizing an amount of energy consumed in this health benefiting implementation. The inventors have further observed that pulse trains may be used on sleeping people to modify their circadian rhythm, without disturbing their sleep architecture.

In some exemplary implementations, dosing determination component 34 may prioritize dosing, e.g., from between emissions that promote sleep health and emissions that promote brain health, effectively providing different recipes of light. These different recipes may be modified using independent parameter sliders on a UI.

In some exemplary implementations, backlight control component 36 and/or panel control component 38 may adjust one or more display properties. For example, after determining the EFR, the display properties may be adjusted within the EFR or in another region (e.g., peripheral, above a viewed horizon, etc.) such that a dosing of one of cyan and LRNE illumination is provided, while the user is gazing at the EFR.

Because color perception and resolution are lower outside of an EFR, some exemplary implementations of backlight component 36 and/or panel component 38 may control display 70 such that true color(s) (i.e., accurate coloring) is not always provided outside of the EFR. For example, color may be tinted towards cyan for more EML outside the EFR. Accordingly, some exemplary implementations of backlight component 36 and/or panel component 38 may control display 70 such that true color is provided within the EFR. As used herein, a description of controlled lighting and/or color outside of the EFR implies a rest of the screen of display 70. For example, the rest of the screen may be kept at one of higher and lower resolution. In another example, the rest of the screen may be kept at one of higher and lower EML. In some exemplary implementations, display 70 may be configured to generate true color within two and ten degrees of a user's cones, but color accuracy elsewhere may be reduced comparatively.

In some exemplary implementations, backlighting 60 may comprise mini (e.g., submillimeter) LEDs, this technology being between micro LED and standard LED technologies. Standard-sized LEDs (e.g., used in LCDs) are about 1,000 microns in size, whereas mini LEDs may be about 200 microns in size. The smaller size of mini LEDs may allow hundreds or even thousands to be placed in blacklight 60, an actual amount depending on a screen size of display 70. Backlighting 60 may send light through the pixels of panel 65, which may contain more detailed image information. Color may additionally be added after the light passes through RGB filters before reaching the screen surface.

In some exemplary implementations, LEDs 62 of backlighting 60 may be brightened or dimmed in small groups in synchronization with the image information of the pixels. Some exemplary implementations of backlight component 36 and/or panel component 38 may control lighting to a low power mode by decreasing intensity and/or EML outside the EFR. One or more of these components may enhance the dynamic range (e.g., blacker blacks, achieving a ratio greater than 90:1 for white to black pixels) of panel 65 by reducing intensity of or by entirely turning off a portion of backlighting 60 that effects pixels of panel 65 intending to display blackness or other darkness. In some exemplary implementations, backlight control component 36 may, when performing this local dimming, modulate the gamut and spectral content in that same portion (e.g., cell 67). In these or other implementations, backlight control component 36 may shut certain infrared-emitting pixels off outside of the EFR (e.g., in the user's peripheral vision), these pixels being otherwise on.

In some exemplary implementations, determination component 34 may determine a dosing having normal RGB for an EFR and CSE for the rest of the screen, effectively controlling different sets of cells differently. For example, long blue light at a high intensity may be determined for cells above a user's horizon, e.g., emitting from a wall panel (i.e., rather than vertical illumination). Since most (e.g., 90% ) of a user's screen is often white, determination component 34 may instead determine a high EML dosing for those screen portions. As used herein, a cell is a collection of pixels that are being illuminated by a different backlight LED 62. For example, each LED 62 may be driven with different parameters (e.g., color, intensity, etc.) at a same time.

Humans do most high resolution seeing with a fairly small part of the eye. Accordingly, some exemplary implementations of backlight component 36 and/or panel component 38 may control display 70 such that a resolution is reduced outside the EFR.

Some exemplary implementations of panel component 38 may control panel 65 such that a jitter is adjusted outside the EFR.

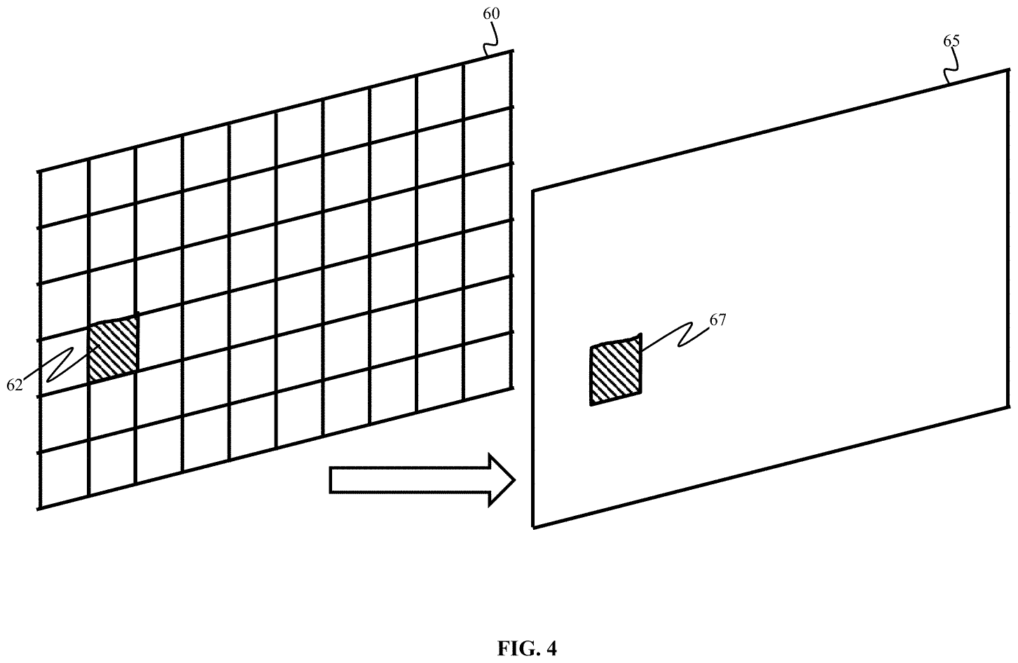

In some exemplary implementations, backlighting 60 may comprise a grid or array of several (e.g., tens, hundreds, or even a few thousands) of LEDs 62, as depicted in FIG. 4. A set of LEDs 62 may form a channel. For example, backlighting 60 may implement one, two, four, six, or n channels, n being any natural number. LEDs 62 may each be substantially smaller than a screen of panel 65 but substantially greater than each of the pixels, each LED having a predetermined one-to-many mapping to a set of pixels. This set of pixels may form cell 67, as depicted in FIG. 4, with each cell having many pixels (e.g., hundreds, thousands, or even a few millions). As such, in a four channel implementation, display 70 may have four LEDs per cell. In some exemplary implementations, backlight control component 36 may be configured to change a spectrum of backlight 60 by driving each channel differently. And the color of each colored pixel may be a function of the transmission spectrum of the filter on that pixel multiplied by the spectrum of the backlight. This multiplication may result in moving the comers of the triangular gamut significantly (e.g., from green and blue comers of the sRGB space to closer to cyan in the chromaticity diagram).

In general, light corresponding to a correlated color temperature (CCT) of about 2,700 kelvin (K) to about 6,500 K is considered to be white light. A white point of 6,500 K is typical for many displays. Different displays may have different white points, e.g., 6,300 K or 6,000 K. In two-channel architectures that white point will always look the same. As mentioned, 90% of the screen may often be white (e.g., in computer displays of office work) so some exemplary implementations of backlight control component 36 and/or panel control component 38 may cause a same look when changing the melanopic content of that white, i.e., without users being able to notice the change. As such, backlight control component 36 and/or panel control component 38 may match the normal color that the users expect out of display 70, while minimizing the amount of noticeable change when shifting between EML modes.

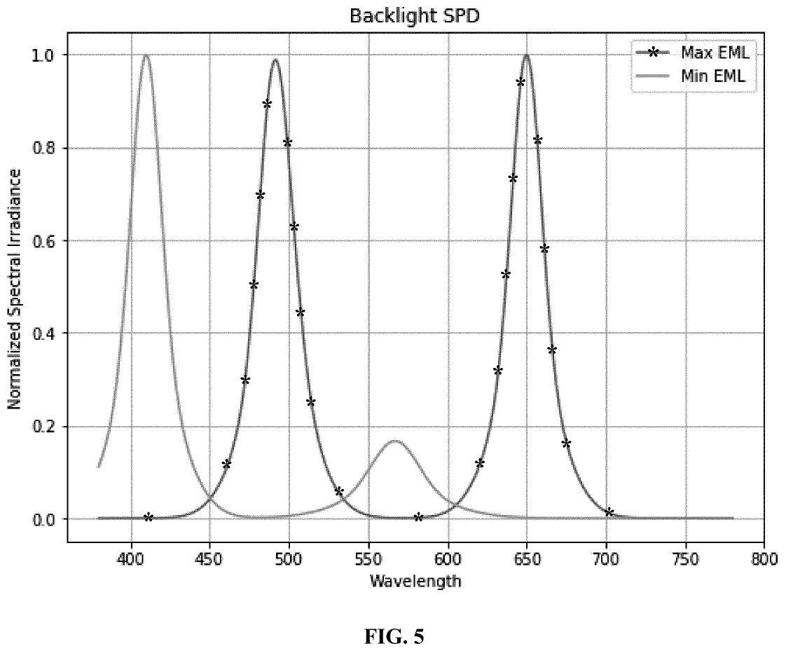

FIG. 5 depicts a backlight SPD for a two-channel driver (e.g., using LED technology but this driver may be otherwise implemented) to generate this spectra. For example, backlight control component 36 may drive one channel 100% and the other channel 0% . In another example, backlight control component 36 may drive these channels with vice versa percentages. And, in another example, backlight control component 36 may drive them with intermediate values (e.g., one channel 90% and the other channel 10% ) by shifting the drive weight from one channel to the other. As such, the disclosed two channel configuration may maximize and minimize a delivered EML, while maintaining a white point at 6,500 K. The white point of panel may be characterized by a chromaticity, all other chromaticities being based on this chromaticity using polar coordinates.

The first lighting channel may comprise an LED having a 450 nm peak wavelength and an associated luminophoric medium having one or more phosphors, quantum dots, or a mixture thereof. The second lighting channel may comprise an LED having a 410 nm peak wavelength and an associated luminophoric medium having one or more phosphors, quantum dots, or a mixture thereof.

In one example for regular (e.g., office) workers, backlight control component 36 may cause a maximum EML at some point during the morning (e.g., between 9 AM and 11 AM). In another example for a night shift worker, backlight control component 36 may be based on the precise timing of this other user's body clock such that the maximum EML is provided later in the day.

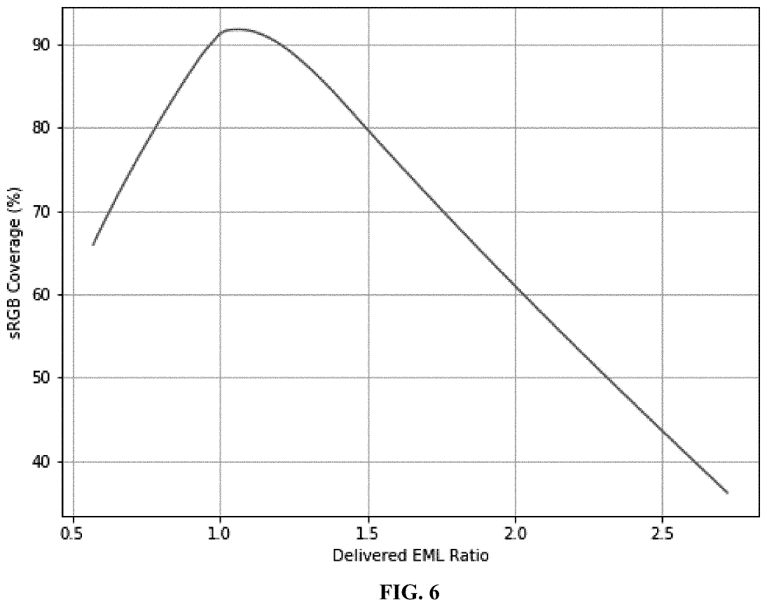

FIG. 6 exemplarily depicts a peak sRGB coverage of nearly 100% with a delivered EML ratio of 1. In some exemplary implementations, backlight control component 36 may thus drive backlight 70 and panel 65 may drive its pixels such that most of the today is at the full sRGB coverage (e.g., from 11 AM to 5 PM). Backlight component 36 and/or panel component 38 may then slowly (e.g., over the course of an hour or two) shift to a low EML mode to prevent suppression of melatonin in the evening for supporting a healthy sleep schedule. These components may achieve higher percentages of sRGB coverage to better reflect what a user should expect from display 70, as if display 70 were merely a typical display. As depicted in FIG. 6, display 70 may achieve a minimum (min)/maximum (max) ratio of almost 1.5, while being capable of almost complete sRGB coverage.

In some exemplary implementations, backlight control component 36 may determine a gamut that is rendered by pixels of panel 65 by adjusting the backlighting spectrum. This adjustment may be performed without adjusting a filter (e.g., on a colored RGB pixel) in one or more portions of panel 65, and it may cause a switch between high EML and low EML modes. In these or other implementations, backlight control component 36 may continually perform adjustments such as this by providing different gamut coverages (e.g., gamut 110) within these two modes. Other implementations may provide gamut coverage in other ranges (e.g., between other mode extremes).

In some exemplary implementations, backlight control component 36 may adjust backlighting 60 such that pixels of panel 65 render a high EML gamut. For example, high EML gamut 108 may be a smaller one of the two extremes (as exemplarily depicted in FIGS. 7A-7F), and in other examples this gamut may be a larger of the two extremes. Accordingly, backlight control component 36 may maximize the EML by determining a smallest gamut that completely contains or encloses all chromaticities (e.g., cluster 104) corresponding to LED 62. This maximum EML gamut may display all colors that are supposed to be displayed in that region of panel 65.

In some exemplary implementations, backlight control component 36 may determine an enclosing gamut by first determining a set of colored pixels (e.g., red, green, and blue) that may be affected by backlight 60. This set of pixels may be represented in matrix form, e.g., with subscripts for each pixel ranging from Ro, Go, and Bo to Rn, Gn, and Bn, n being any natural number. System 10 may thus be configured to perform triangulation to determine what values to drive a pixel based on a certain backlight to preserve the target pixel color. This preservation of the output chromaticity may be accomplished, e.g., using a matrix operation.

The enclosing gamut may be determined via a suitable algorithm. For example, some implementations may perform a convex hull algorithm. In another example, panel control component 38 may project the pixels into an angular representation of a color space with an origin focused around the red corner (e.g., at the lower, right corner of FIG. 2). This red corner is not expected to appreciably move. Panel control component 38 may then put a line between the middle of the shortest edge of triangle 108 and that red corner, which would be the zero direction, and then panel control component 38 may project the line from the right-hand corner to each color to determine the angle between that and the zero line. As such, the maximum and minimum angles would be indicative of the gamut.

In some exemplary implementations, backlight control component 36 may determine parameters of a spectrum from backlight 60 based on the determined, enclosing gamut. Panel 65 may drive each colored pixel. But, to set a particular color, panel 65 may take into account the target color in sRGB space, the backlight spectrum having been changed. For example, panel 65 may intend a pixel to display pink based on a mapping between backlighting parameters and pixel display parameters. That is, panel 65 may drive the pixel to purple such that effect of the backlight causes pink to be displayed at that pixel. Accordingly, some implementations of backlight control component 36 may translate an input RGB to a local RGB such that panel control component 38 subsequently sets the pixels of panel 65.

In some exemplary implementations, backlight control component 36 may determine a gamut to-be-rendered by pixels of panel 65, by adjusting the backlighting spectrum. This adjustment may be performed without adjusting a filter (e.g., on a colored RGB pixel) in one or more portions, and it may cause a switch between high EML and low EML modes. Backlight control component 36 may continually perform adjustments such as this by providing different gamut coverages within these two modes. Other implementations may provide gamut coverage in other ranges (e.g., between other mode extremes). In some exemplary implementations, backlight control component 36 may choose the spectrum on a per cell basis based on whether the user is looking at that portion of the screen. And an amount of distortion away from that chosen spectrum may be continually greater the farther a pixel is from that EFR.

In some exemplary implementations, backlight control component 36 may adjust one or more display properties in a region outside of the EFR by adjusting the backlighting spectrum such that a color of each pixel in the region is distorted away from an originally determined color to one closer to cyan or long-red NIR (LRN) and that an intensity of the each pixel is decreased.

In some exemplary implementations, backlight control component 36 may adjust backlighting 60 such that pixels of panel 65 render a high EML gamut, which in some examples is a smaller of the two extremes and in other examples is a larger of the two extremes. Accordingly, backlight control component 36 may maximize the EML by determining a smallest polygon (e.g., triangular high EML gamut 108) that completely contains or encloses all chromaticities of a color space (e.g., cluster 104) that correspond to LED 62. This maximum EML gamut may display all colors that are supposed to be displayed in that region of panel 65

In some exemplary implementations, backlight control component 36 may make gamut decisions in real time on a per region or per cell basis. Each region may have an appropriate pairing of color mapping at the pixel level and backlight setting to produce accurate colors, while optimizing the EML according to the desired output of the whole system. That is, backlight control component 36 may make appropriate pairings of color mappings at the pixel level and the backlight setting. For example, a GPU may determine a certain chromaticity for a pixel, and then panel 65 may determine individual RGB pixel transmission levels to meet that chromaticity, given that the RGB chromaticities are not the traditional RGB chromaticities of an sRGB monitor. The calculations that are done in the GPU may be based on the assumption that the RGB chromaticities are substantially the comers of sRGB space. For example, to achieve a certain blue an implementation of panel 65 would need a certain amount of blue, a certain amount of green, and a certain amount of red. If panel 65 moves the comers of the gamut, then in order to achieve that color that the GPU intended, instead of using the weights that the GPU transmitted as an RGB coordinate or RGB vector, then panel control component 38 may need to perform a mapping because the gamut has been distorted. That is, in this example, panel control component 38 may need to set pixels such that each of RGB transitions to a certain level that has been changed because the blacklight has changed as well. As such, backlight control component 36 and panel control component 38 may coordinate function so that what the user sees matches what is expected based on the RGB coordinates sent out from the GPU, across the whole screen of panel 65.

FIGS. 7A-7F exemplarily depict a changing gamut coverage, as backlight control component 36 adjusts the weighting between high and low EML modes. In this example, FIG. 7A depicts an initial, highest EML mode, and FIG. 7F depicts a final, lowest EML mode (but this is not intended to be limiting as a low EML mode may be first with the high EML mode being last). This gamut coverage represents different color performances of the different modes. For example, the high EML mode, having saturated blues and greens, may transition through a triangle representing good sRGB coverage until the color space shifts towards violets and yellows and oranges as gamut 310 transitions into a lower EML mode. FIG. 7 depicts a two channel backlight configuration, but a similar example may be demonstrated with a four-channel backlight configuration (e.g., FIG. 11); the four-channel backlight configuration may produce a better gamut. The two-channel system (e.g., two LEDs instead of four) may be simpler (e.g., in algorithmic decision making for proper blending of channels to set a correct color) and occupy less space of backlighting 60 to perform the disclosed color mixing. Implementations of backlight 60 with a four-channel driver may perform better (e.g., in terms of better sRGB coverage and of a greater range in EML extremes, while having greater flexibility).

In FIGS. 7A-7F are depicted sRGB gamut 302, maximum EML gamut 308, minimum EML gamut 307, intermediate spectra gamut 310, and covered region within sRGB 315. As depicted in FIG. 7A, intermediate gamut 310 may shrink to converge (and thus capable of overlapping) with gamut 308 for one extremity. Similarly, as depicted in FIG. 7F, intermediate gamut 310 may grow to converge (and thus capable of overlapping) with gamut 307 for the other extremity.

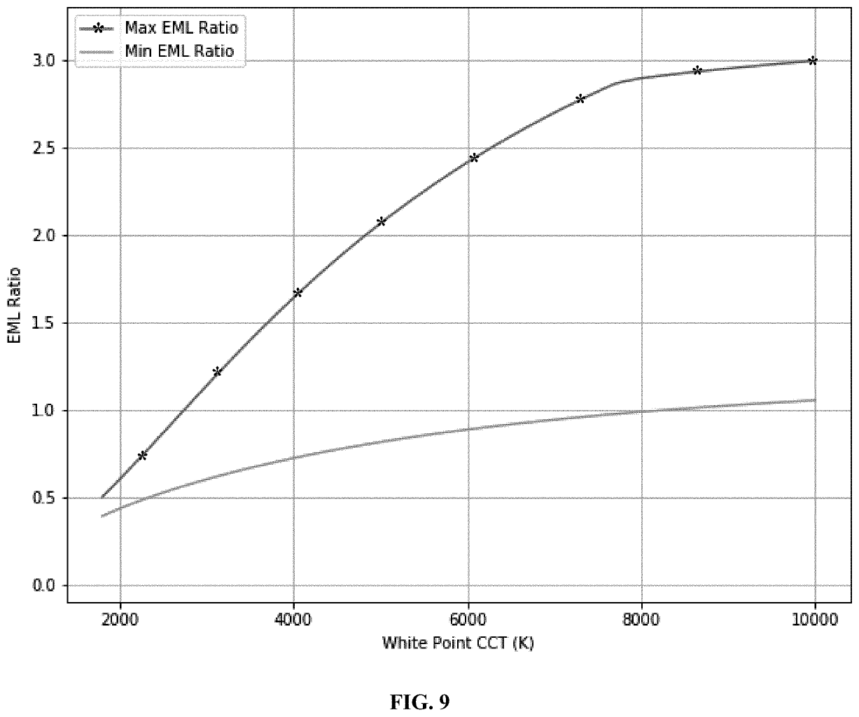

Similar to FIG. 5, FIG. 8 also exemplarily depicts an SPD of backlight 60 but instead when backlight 60 is configured to have four channels. As such, the disclosed four channel configuration may maximize and minimize a delivered EML, while maintaining a white point at 6,500 K. FIGS. 5 and 8 may be used to demonstrate that backlight control component 36 may adjust the SPD of backlight 60 to positively affect the human circadian system. This adjustment may include modulating an amount, spectrum, timing, and duration of exposure of the user to this light. In another exemplary adjustment, backlight control component 36 may adjust a white point of the backlighting spectrum, while preserving chromaticities of pixels of the surface.

FIG. 11 exemplarily depicts a four-channel LED chromaticity diagram. More particularly, in this figure is depicted sRGB gamut 302′, minimum EML gamut 307′, intermediate spectra gamut 310′, and covered region within sRGB 315′.

As is demonstrable with respect to FIG. 9, backlight control component 36 may determine a white point temperature. This determination may be automatically selected or based on user input (e.g., via UI device 18). The white point temperature is typically static on conventional displays, or it may be marginally adjusted with software filters. But the disclosed backlight may adjust white point, e.g., while preserving all 16 million accessible colors. But backlight control component 36 may cause a wide range between high and low EML modes at that temperature. For example, backlight control component 36 may cause a high EML in the morning at 10,000 K with a ratio of 3, and then slowly bring the white point temperature down to 6,000 K between 11 AM and 12 PM, and then between 12 PM and an end of a business day the temperature may stay the same, while traversing the EML ratio all the way down the minimum EML ratio line. Continuing with this example, backlight control component 36 may then, as the day progresses into the afternoon and evening, cause a traversal of that minimum EML ratio line off to the left to the warmer white point temperatures and lower EML ratio. In the example of FIG. 9, a ratio of maximum EML at 6,500 K to minimum EML at 2,500 K is 2.56/0.53, which is 4.88. In this or another example, backlight control component 36 may output a lower intensity in the early morning, transition to a higher intensity as the day progresses, and reduce to a lower intensity in the evening.

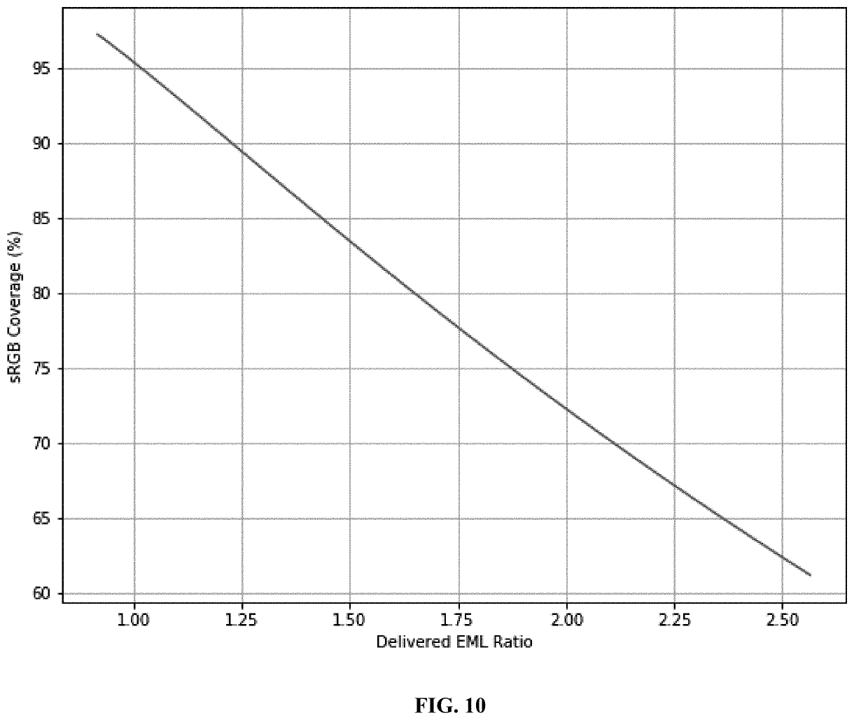

FIG. 10 exemplarily depicts a mini LED implementation where high EML regions have a smaller gamut coverage. In some exemplary implementations, backlight control component may cause panel 65 to show whites with high EML, and the more saturated colors that are requiring that good gamut coverage may have to have a lower EML gamut. Accordingly, the saturated reds and blues may require a lower EML in this region having these saturated colors.

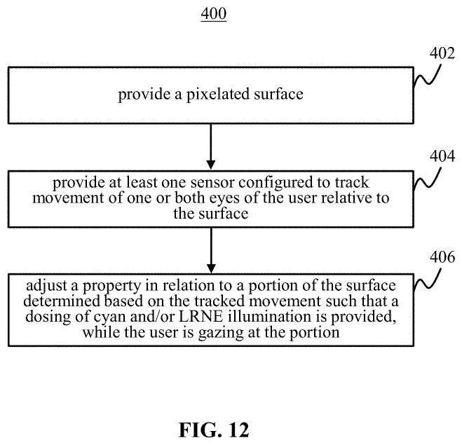

FIG. 12 illustrates method 400 for controlling light based on a user's gaze, in accordance with one or more exemplary implementations. Method 400 may be performed with a computer system comprising one or more computer processors and/or other components. The processors are configured by machine readable instructions to execute computer program components. The operations of method 400 presented below are intended to be illustrative. In some exemplary implementations, method 400 may be accomplished with one or more additional operations not described, and/or without one or more of the operations discussed. Additionally, the order in which the operations of method 400 are illustrated in FIG. 12 and described below is not intended to be limiting. In some exemplary implementations, method 400 may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of method 400 in response to instructions stored electronically on an electronic storage medium. The processing devices may include one or more devices configured through hardware, firmware, and/or software to be specifically designed for execution of one or more of the operations of method 400.

At operation 402 of method 400, a pixelated surface may be provided. As an example, the surface may be an LCD panel that is complemented with a backlighting spectrum provided by an array of LEDs 62. In some exemplary implementations, operation 402 is performed by obtaining or manufacturing panel 65 (shown in FIG. 1 and described herein).

At operation 404 of method 400, at least one sensor configured to track movement of one or both eyes of the user relative to the surface may be provided. As an example, eye sensor 50 may generate output signals such that an EFR of a user is determined. In some exemplary implementations, operation 404 is performed by a processor component the same as or similar to evaluation component 32 (shown in FIG. 1 and described herein).

At operation 406 of method 400, a display property may be adjusted in relation to a portion of the surface determined based on the tracked movement such that a dosing of cyan and/or LRNE illumination is provided, while the user is gazing at the portion. For example, evaluation component 32 may measure a duration that a user has been gazing at the surface so that backlight control component 36 and/or panel control component 38 may adjust one or more display properties based on this measurement. These properties may include at least one of wavelength, duration, SPD, and intensity. In another example, backlight component 36 may control one or more properties of the backlighting spectrum based on a gamut that ranges between a first gamut causing a first amount of EML and a second gamut causing a second amount of EML greater than the first amount. In some exemplary implementations, operation 406 is performed by a processor component (e.g., as shown in FIG. 1 and described herein).

Techniques described herein can be implemented in digital electronic circuitry, or in computer hardware, firmware, software, or in combinations of them. The techniques can be implemented as a computer program product, i.e., a computer program tangibly embodied in an information carrier, e.g., in a machine-readable storage device, in machine-readable storage medium, in a computer-readable storage device or, in computer-readable storage medium for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps of the techniques can be performed by one or more programmable processors executing a computer program to perform functions of the techniques by operating on input data and generating output. Method steps can also be performed by, and apparatus of the techniques can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, such as, magnetic, magneto-optical disks, or optical disks. Information carriers suitable for embodying computer program instructions and data include all forms of non-volatile memory, including by way of example semiconductor memory devices, such as, EPROM, EEPROM, and flash memory devices; magnetic disks, such as, internal hard disks or removable disks; magneto-optical disks; and CD-ROM and DVD-ROM disks. The processor and the memory can be supplemented by, or incorporated in special purpose logic circuitry.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed:

1. A method comprising:
providing a pixelated surface;
providing at least one sensor configured to track movement of at least one eye of a user relative to the surface;
detecting one or more portions of the surface based on the tracked movement; and
adjusting one or more properties in relation to the one or more detected portions such that a dosing of one of cyan and long-red, near-infrared (NIR) illumination is provided, while the user is gazing at the one or more portions.

2. The method of claim 1, further comprising:
determining a current time, a current date, a current geolocation of the surface at a time of the gazing, and an estimated body clock of the user,
wherein the one or more properties are adjusted based on the determination(s).

3. The method of claim 1, further comprising:
measuring a duration that the user has been gazing at the surface,
wherein the one or more properties are adjusted based on the measurement.

4. The method of claim 1, further comprising:
measuring, via at least one of a user-attached sensor and at least one remote sensor, at least one of a physical activity, an ambient noise, a hormonal level, and an insulin level, with respect to the user,
wherein the one or more properties are adjusted based on the measuring satisfying one or more criteria, while the user is gazing at the one or more portions.

5. The method of claim 1, further comprising:
measuring, via at least one of a user-attached sensor and at least one remote sensor, at least one of an exposure to a cyan wavelength over at least one first time frame and an exposure to a long-red, NIR wavelength over at least one second time frame, with respect to the user,
wherein the one or more properties are adjusted based on the measuring satisfying one or more criteria, while the user is gazing at the one or more portions.

6. The method of claim 1, wherein the one or more adjusted properties are at least one of wavelength, duration, spectral power distribution (SPD), and intensity.

7. The method of claim 1, wherein the surface is one of a monitor, a handheld display, a wearable glasses, an augmented reality (AR) screen, and a virtual reality (VR) screen.

8. The method of claim 1, wherein the surface is a liquid-crystal display (LCD) complemented with a backlighting spectrum provided by an array of light-emitting diodes (LEDs), one or more properties of the backlighting spectrum being based on a gamut that ranges between a first gamut causing a first amount of equivalent melanopic lux (EML) and a second gamut causing a second amount of EML greater than the first amount, and wherein each gamut completely encloses all chromaticities within a polygon on a color space.

9. The method of claim 8, further comprising:
determining a mapping between the one or more properties of the backlighting spectrum and the one or more properties related to the one or more portions, the mapping being used to meet a target color for each pixel in the one or more portions.

10. The method of claim 8, wherein a predetermined amount of pixels of the surface corresponds with each LED of the array, the chromaticities representing a set of colored pixels corresponding to one LED of the array.

11. The method of claim 8, further comprising: adjusting the backlighting spectrum without adjusting a filter on a colored pixel in the one or more portions.

12. The method of claim 11, wherein the adjustment of the backlighting spectrum adjusts a white point while preserving chromaticity of pixels of the surface.

13. The method of claim 8, wherein the ranging of the gamut between the first gamut and the second gamut is performed by adjusting a drive weight of two or more channels of the array.

14. The method of claim 4, wherein the user-attached exposure sensor is one of a pendant, dongle, an activity tracker, and a tag, and wherein the at least one remote exposure sensor is a room sensor.

15. The method of claim 4, wherein the physical activity comprises steps walked by the user, at least one of a heart rate of the user, and a blood pressure of the user, wherein the ambient noise is measured in decibels, a respective criterion being a noise threshold above which stress increases and sleep is disrupted, and wherein the hormonal level is based, at least in part, on stress-inducing cortisol.

16. The method of claim 1, wherein the one or more properties are adjusted in a region outside of the one or more portions by adjusting one or more properties of backlighting spectrum such that (i) a color of each pixel in the region is distorted away from an originally determined color to one closer to cyan or long-red NIR (LRN) and (ii) an intensity of the each pixel is decreased.

17. The method of claim 16, wherein an amount of the distortion is greater the farther a pixel is from the one or more portions.

18. The method of claim 1, wherein the one or more adjusted properties comprise provision of a light pulse train.

19. A system, comprising:
a pixelated surface;
at least one sensor configured to track movement of one or both eyes of a user relative to the surface;
a non-transitory recording medium including instructions for impacting biological activity of the user; and
a processor operably coupled to the recording medium for executing the instructions of:
detecting one or more portions of the surface based on the tracked movement; and
adjusting one or more properties in relation to the one or more detected portions such that a dosing of one of cyan and long-red, near-infrared (NIR) illumination is provided, while the user is gazing at the one or more portions.

20. A non-transitory computer-readable medium comprising instructions executable by at least one processor to perform a method, the method comprising:
providing a pixelated surface;
providing at least one sensor configured to track movement of one or both eyes of the user relative to the surface;
detecting one or more portions of the surface based on the tracked movement; and
adjusting one or more properties in relation to the one or more detected portions such that a dosing of one of cyan and long-red, near-infrared (NIR) illumination is provided, while the user is gazing at the one or more portions.
